(19) United States
(12) Patent Application Publication
(10) Pub. No.: US 2008/0031400 A1
(43) Pub. Date: Feb. 7, 2008

(54) 3D Localization Of Objects From Tomography Data

(76) Inventors: Luc Beaulieu, Promenade des Soeurs (CA); Dragan Tubic, Charlesbourg (CA)

Correspondence Address: OGILVY RENAULT LLP, 1981 MCGILL COLLEGE AVENUE, SUITE 1600, MONTREAL, QC, H3A2Y3, CA

(21) Appl. No.: 11/579,728

(22) PCT Filed: May 6, 2005
(86) PCT No.: PCT/CA05/00700

§ 371 Date: Nov. 6, 2006

Related U.S. Application Data
(60) Provisional application No. 60/568,256, filed on May 6, 2004.

(51) Int. Cl. A61B 6/03

(52) U.S. Cl. 378/4

(57) ABSTRACT

Unlike existing methods for three-dimensional seed reconstruction, the proposed method uses raw tomography data (sinograms) instead of reconstructed CT slices. The method is for three-dimensional reconstruction of an object inserted in a living or non-living body. It comprises obtaining raw tomography data for an area of the body where the object is inserted; detecting a trace of the object in the raw tomography data, by extracting points from the trace; and estimating at least one of a position and an orientation of the object using the points and a known shape of a trace of the object in the raw tomography data.

### CROSS-REFERENCE TO RELATED APPLICATIONS

This application claims priority of U.S. provisional patent application No. 60/568,256 filed on May 6, 2004 by Applicants, the contents of which are hereby incorporated by reference.

### TECHNICAL FIELD

The invention relates to three-dimensional localization of objects in raw tomography data. More specifically, it relates to the localization of objects using sinograms from tomographic image acquisition procedures.

### BACKGROUND OF THE INVENTION

Medical equipment for radiation therapy treats tumorous tissue with high energy radiation. The amount of radiation and its placement must be accurately controlled to ensure both that the tumor receives sufficient radiation to be destroyed, and that the damage to the surrounding and adjacent non-tumorous tissue is minimized.

Three-dimensional reconstruction of prostate implants follows the brachytherapy from its beginnings. Starting with radiographic films, the reconstruction changed with imaging trends from the use of radiographic films to fluoroscopic images and finally computed tomography (CT). Being widely available, cost effective and fast, CT imaging has become the main imaging tool used for implant reconstruction.

A common point to all three dimensional (3D) reconstruction algorithms is the use of reconstructed CT images (slices). All algorithms proceed by first thresholding (converting a CT slice into a binary image, where white pixels indicate objects of interest) each slice according to a pre-specified threshold value that corresponds to the Hounsfield Unit (HU) numbers of seed material. After this step, each slice contains connected components or “blobs” (groups of connected pixels) that represent detected seeds. Since the 3D position of each pixel is known, the 3D reconstruction is reduced to the resolution of the following two problems. First, a single seed can be present on more than one slice and the components on different slices have to be grouped together in order to estimate the correct number of seeds. Second, the closely spaced seeds can appear connected and contained in a single component. Such components then have to be broken into single seeds.

The main difficulty in resolving the above problems comes from the fact that the seeds are severely undersampled in the scanning direction. This is a consequence of the inter-slice distance which is very large with respect to the length of a seed: typically 1.5-5 mm whereas the seed length is 4.5-5 mm. For large interslice distances, it is sometimes impossible to decide if two components observed in two subsequent slices belong to a single or to separate seeds, as shown in FIG. 1.

FIG. 1 is an example of ambiguities in thresholded CT images. After thresholding, pixels in CT slices are separated into two groups. The first group contains pixels belonging to the background (textured portions) and the other one contains pixels that belong to seeds (white portions). Due to the interslice distance, ambiguities arise and a unique solution cannot be found. FIG. 1a is the observed thresholded slices. FIGS. 1b and 1c are two example configurations of seeds that might have generated the two slices of FIG. 1a.

As mentioned above, detected components can contain more than one seed due the small distance between seeds, but also due to the reconstruction artifacts. Being metallic, the seeds represent discontinuities of the density and thus create artifacts in CT images. Thresholding such images connects closely spaced seeds resulting in detected components containing more than one seed. The artifacts also appear if the patient has metallic implants (in the hip, for example) in which case the reconstruction from CT images is difficult, as illustrated in FIG. 2.

In FIG. 2, artifacts produced by metallic objects are shown. In FIG. 2a, the reconstructed CT slice for a patient with a hip prosthesis is shown. In FIG. 2b, the corresponding sinogram for the same patient is shown. While the reconstructed CT image is heavily corrupted and almost unusable, the sinogram is artifacts free.

Breaking the components in single seed is not an easy task, once again due to the undersampling problem. For example, it is very difficult to decide whether the components in FIG. 1 represent two seeds perpendicular to CT images or almost parallel to it. Finally, the position of the seeds is difficult to estimate accurately, especially with respect to the orientation of the seeds. Even with the most recent automated CT detection algorithms, localization errors remain important for a fraction of the seeds. Liu et al quote maximum errors of 5 to 7 mm and an average error of 2.7 to 3.1 mm in their article entitled “Automatic localization of implanted seeds from post-implant CT images” published in 2003 in Physics in Medicine & Biology. In comparison, the best automated film-based (or fluoroscopy-based) algorithms give average errors of usually less than 1 to 1.5 mm.

### SUMMARY OF THE INVENTION

Accordingly, an object of the present invention is to overcome drawbacks of the prior art.

Another object of the present invention is to localize 3D objects in raw tomography data.

Unlike existing methods for three-dimensional seed reconstruction, the proposed method uses raw tomography data (sinograms) instead of reconstructed CT slices. The method is for three-dimensional reconstruction of an object inserted in a living or non-living body. It comprises obtaining raw tomography data for an area of the body where the object is inserted; detecting a trace of the object in the raw tomography data, by extracting points from the trace; and estimating at least one of a position and an orientation of the object using the points and a known shape of a trace of the object in the raw tomography data.

According to a first broad aspect of the present invention, there is provided a method for three-dimensional reconstruction of an object inserted in one of a living and a non-living body, a material of said object providing sufficient contrast with respect to a material of said body in raw tomography data, the object having one of a point-like shape and a curvilinear shape. The method comprises obtaining raw tomography data for an area of the body where the object is inserted; detecting a trace of the object in the raw tomography data, by extracting points from the trace; and estimating at least one of a position and an orientation of the object using the points and a known shape of a trace of the object in the raw tomography data.

According to a second broad aspect of the present invention, a system for three-dimensional reconstruction of an object inserted in one of a living and a non-living body, a material of said object providing sufficient contrast with respect to a material of said body in raw tomography data, the object having one of a point-like shape and a curvilinear shape. The system comprises a scanner for obtaining raw tomography data for an area of the body where the object is inserted; a trace detector for detecting a trace of the object in the raw tomography data, by extracting points from the trace; and an object locator for estimating at least one of a position and an orientation of the object using the points and a known shape of a trace of the object in the raw tomography data.

In this specification

the term “sinogram” is intended to mean an image representation of data obtained when projection-reconstruction imaging is used;

the term “computed tomography (CT)” is intended to mean the general process of creating cross-sectional or tomographic images from projections (line integrals) of the object at multiple angles and using a computer for image reconstruction;

the term “raw tomography data” is intended to mean the data collected during the process of using motion of a focal spot and image receptor (e.g. film) in generating tomographic images where object detail from only one plane or region remains in sharp focus. Details from other planes in the object which would otherwise contribute confounding detail to the image, are blurred and effectively removed from visual consideration in the image;

the term “implant” and the term “seed” are intended to be synonyms and are intended to mean any foreign body implanted into a patient for therapeutic or cosmetic reasons;

the term “body” is intended to include a living or a non-living body;

the term “slice” is intended to mean a section to be imaged during the imaging process in a cross-sectional imaging modality; and

the term “blob” is intended to mean a group of connected pixels which can come from multiple individual objects.

In the present application, three-dimensional reconstruction of prostate seed implants is discussed. Unlike existing methods for implant reconstruction, the proposed algorithm uses raw tomography data (sinograms) instead of reconstructed CT slices. Using raw tomography data solves several inevitable problems related to the reconstruction from CT slices. First, the sinograms are not affected by reconstruction artifacts caused by metallic objects and seeds in the patient body. Secondly, the scanning axis is not undersampled as in the case of CT slices. Moreover, the shape of a single seed in a sinogram can be exactly modeled thus facilitating the detection. All this allows very accurate 3D reconstruction of both position and the orientation of the seeds.

The overall preferred reconstruction procedure is summarized as follows: First, acquisition of the sinogram, that is, raw tomography data, is carried out by the scanning device. Detection and estimation of the curves in the sinogram is done by the seed detector to obtain detected curves. The Hough transform is then computed using the detected curve points and outputs an accumulator. The next main step is thresholding the Hough Transform accumulator in order to obtain blobs representing seeds. These blobs are candidate seed points detected in the accumulator. Back projection of each pixel in each blob to the sinogram is then carried out to obtain projected points, a matching of the projected curve against the detected curves is done and generates matched points, the parameters representing seed position and orientation are computed from the matched points and the steps of projecting, matching and computing are iterated until convergence. Finally, coincident seeds (having similar positions) are grouped together and declared to be a single seed. The final set of seed orientations and positions is then obtained.

### BRIEF DESCRIPTION OF THE DRAWINGS

Further features and advantages of the present invention will become apparent from the following detailed description, taken in combination with the appended drawings, in which:

FIG. 1 is a photograph and comprises FIG. 1a, FIG. 1b and FIG. 1c and shows one example of ambiguities in thresholded CT images. FIG. 1a is the observed thresholded slices. FIG. 1b and FIG. 1c are two example configurations of seeds that might have generated the two slices.

FIG. 2 comprises FIG. 2a and FIG. 2b and shows artifacts produced by metallic objects. FIG. 2a is the reconstructed CT slice for a patient with a hip prosthesis. FIG. 2b is for the corresponding sinogram for the same patient.

FIG. 3 shows a perspective schematic view of tomography scanner geometry.

FIG. 4 shows a schematic plan view of a tomography scanner geometry.

FIG. 5 is a photograph and shows a sinogram of three coplanar seeds spaced by 0.8 mm.

FIG. 6 comprises FIG. 6a, FIG. 6b, FIG. 6c, FIG. 6d, FIG. 6e and FIG. 6f. FIG. 6a shows an ideal curve in an image which has a bar-shaped profile in the direction of its normal. FIG. 6b shows the bar profile, FIG. 6c shows a Gaussian filter, FIG. 6d shows the filtered and convolved function with a well defined maximum; FIG. 6e shows the first derivative and FIG. 6f shows the second derivative.

FIG. 7 is a photograph and is an example of detected curvilinear structures in a sinogram.

FIG. 8 is a photograph and shows the principle of the Hough transform for the seed detection.

FIG. 9 shows the curve detection algorithm tuned to detect only curvilinear structures (whose profile is bar shaped) in a sinogram.

FIG. 10 is a photograph and is an example of the accumulator.

FIG. 11 shows the detection of the position of the seed along the scanning axis. The upper diagram shows the value A(x,y) of a pixel (x,y) in the accumulator. Sₜ(x,y) indicates the presence of seed and it is obtained by thresholding A. The bottom diagram S_{H}(x,y) shows that the obtained region is not sensitive to the oscillations due to the noise.

FIG. 12 shows the Hysteresis threshold transfer function.

FIG. 13 is a photograph and shows the projection of each point in the blob back into the sinogram.

FIG. 14 shows the results of projecting each point in the blob back to the sinogram and matching the resulting trace with the detecting one. Initial points in blobs are indicated as crosses while their final position is shown as a circle.

FIG. 15 is a photograph and shows the traces of oriented seeds. It comprises FIG. 15a and FIG. 15b. FIG. 15a shows the contrast enhanced sinogram for seven seeds with different orientations. Traces, from top to bottom, belong to seeds whose angles with the Z-axis are 90, 0, 15, 30, 45, 60 and 75 degrees. FIG. 15b shows the results of detection.

FIG. 16 is an illustration of the phantoms used for the evaluation of the proposed reconstruction technique. It comprises FIG. 16a, FIG. 16b, FIG. 16c and FIG. 16d. The first phantom consisting of 16 seeds arranged in a regular rectangular 4×4 grid is illustrated in top-view in FIG. 16a. FIG. 16b is a side-view of FIG. 16a. The second phantom having 7 seeds arranged in a single row with equal inter seed distance is shown in FIG. 16c. Each seed is rotated by 15 degrees relative to its neighbouring seed as illustrated by the side-view of the phantom in FIG. 16d.

FIG. 17 is a flow chart of the main steps of the preferred reconstruction method of the present invention.

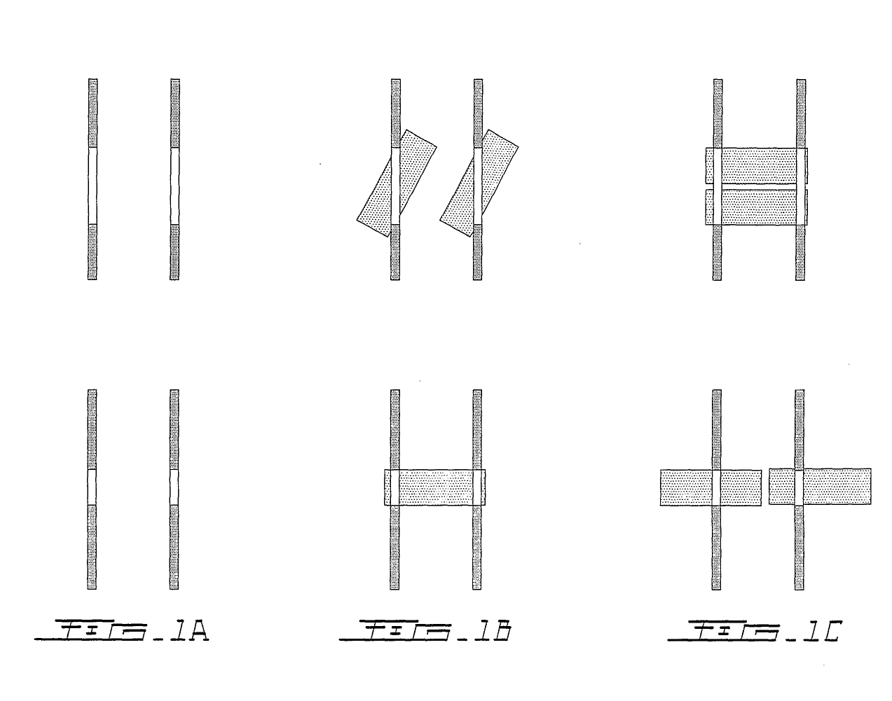

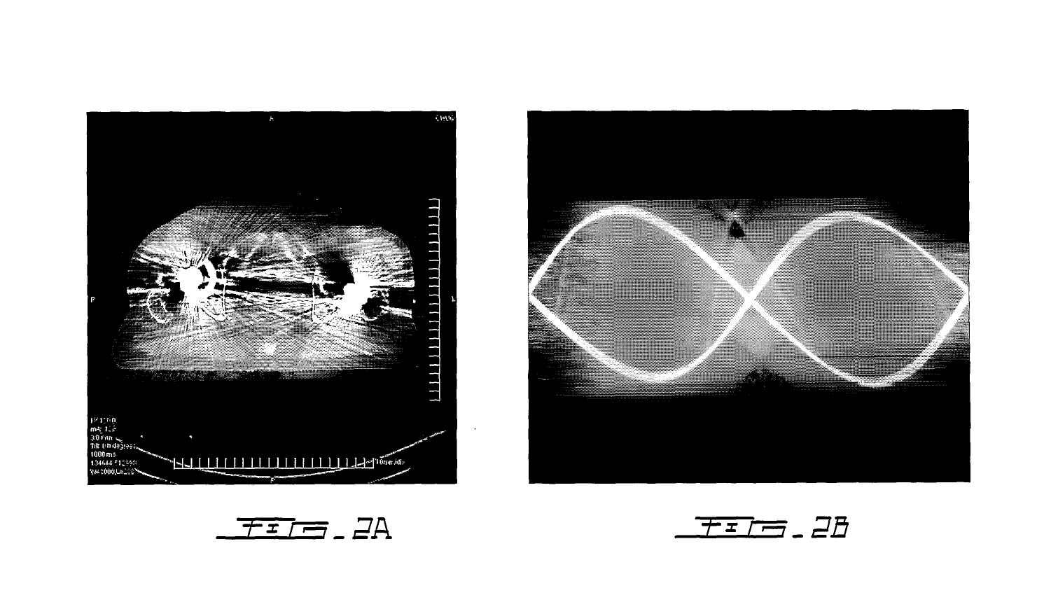

### DETAILED DESCRIPTION OF THE PREFERRED EMBODIMENT

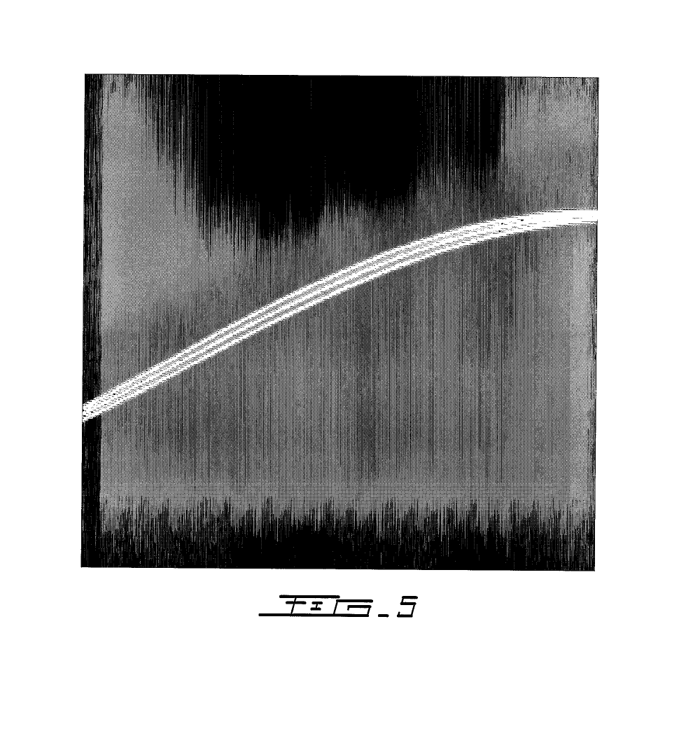

As a solution to the seed reconstruction problem of the prior art, the present invention is based on the reconstruction from the raw tomography data (sinograms) directly. In sinograms, a single seed is typically represented with several hundred samples. This allows reconstruction with unprecedented accuracy: in a test implementation, the position of seeds have been reconstructed with a maximal error of 0.45 mm. Furthermore, sinograms are artifacts free which makes the task of seed detection much easier with respect to the existing approaches. For example, FIG. 5 shows the sinogram for three closely spaced seeds whose “sines” can be easily distinguished. Moreover, the proposed approach is general enough to be used for the reconstruction of any point-like, linear or curvi-linear object that leaves a trace in the sinogram.

Principle of the reconstruction

Scanner Geometry

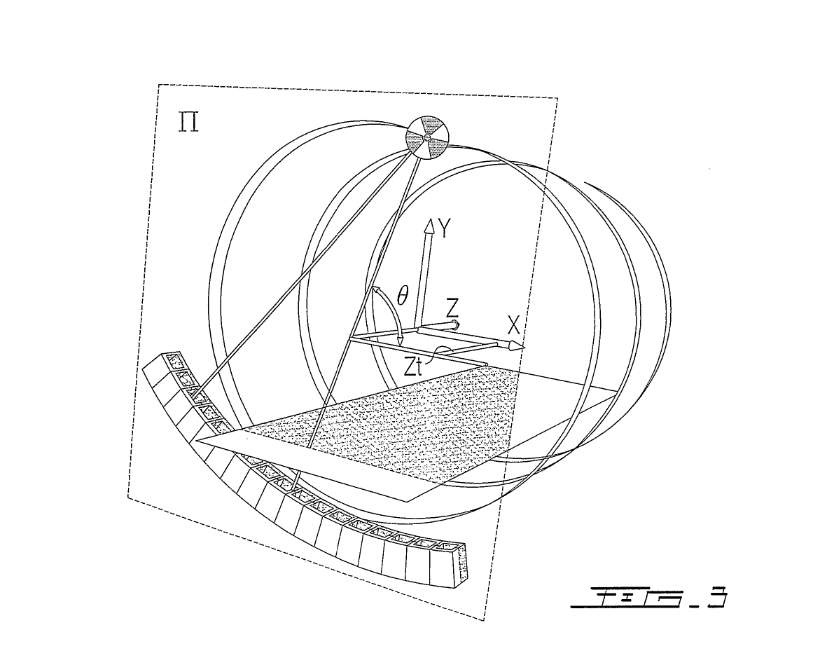

Being the most widely spread, only the helical scanner geometry will be discussed herein. This, however, does not reduce the generality of the proposed approach and how to adopt it for a different geometry will be readily understood by one skilled in the art. As illustrated in FIG. 3, the spiral tomography scanner uses a fan beam of x-rays to measure the attenuation of the patient's body. Both the detector array and the x-ray source are contained in a single plane Π and rotate around the patient (Z axis). Simultaneously, the patient table moves which has the net effect that the detector and the x-ray source move in a spiral path as shown in FIG. 3. The data is typically acquired 1000 times for a single 360 degrees rotation.

The plane Π that contains the x-ray tube and the detector array is assumed perpendicular to the scanning axis Z. Since the axis Z is also the rotation axis, the state of the scanner can be described in terms of the position zₜ of the plane Π along the Z axis as well as the rotation angle θ. The angle θ is the angle between X-axis and the line that connects the x-ray source and the center of the detector. Since the rotation angle θ and the position zₜ are linearly related as θ=2πzₜ/k, the state of the scanner is determined by the angle θ alone. The value of k denotes the “pitch” i.e. the table displacement for a single rotation. The distance from the source to the rotation axis Z is denoted “SAD” (source to axis distance).

Data Acquisition

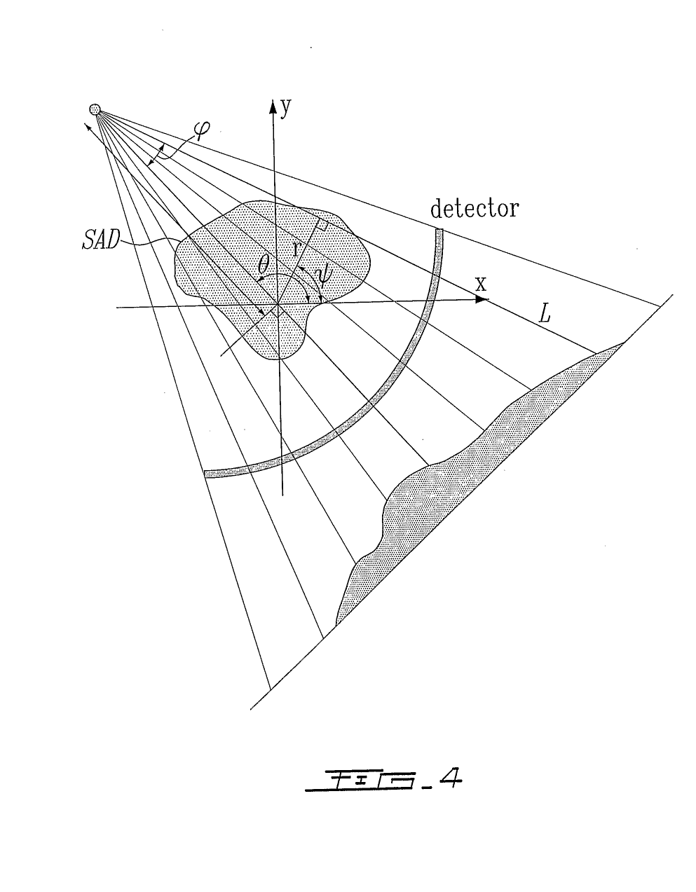

The projection data, i.e. the quantity measured by one detector, is the line integral of the attenuation coefficients along the line L that connects the x-ray source and the detector. For a monochromatic x-ray beam, the projection value for a single ray is defined as follows. Let I₀ be the intensity of a x-ray L at the source. Its intensity I_{d} at the detector is given as:

$${I}_{d} = {I}_{0} exp [ - {\int }_{L} μ ( x , y , z ) ⅆ l ] , \qquad ( 1 )$$

where μ:R³→R defines the attenuation at each point of the 3D space. It is usually assumed that the attenuation is zero everywhere except in a region between the X-ray source and the detector, for example the shaded region in FIG. 4. Furthermore, it is assumed that the same region can be observed, as a whole, from all viewpoints in order to correctly reconstruct an image. This constraint, however, does not apply to the present discussion except that seeds have to be observed.

The projection value, i.e. the attenuation along the line (ray) L is given as:

$${d}_{p} ( L ) = ln \frac{{I}_{0}}{{I}_{d}} = {\int }_{L} μ ( x , y , z ) ⅆ l$$

The parametric representation of the ray L (see FIG. 3) is:
r=x cos(ψ)+y sin(ψ), z=zₜ, (2)

where r denotes the distance between the line and the origin, while ψ denotes its orientation as illustrated in FIG. 4. As mentioned earlier, the X-ray tube and the detector are in a single plane perpendicular to the scanning axis Z. The position of this plane along the Z-axis is denoted zₜ.

Since the detectors are arranged in a regular array along an arc of a circle, the distance r does not change linearly with the position of individual detectors within the array. It is thus more convenient to define the rays in terms angles φ and θ instead of the distance r and the angle ψ. For the same reason, the sinograms from tomography scanners are usually defined using those two angles. The angle φ denotes the relative position of the ray within the fan (see FIG. 4). It is the angle between the ray L and the central ray, i.e. the ray passing through the x-ray source and the origin.

The distance r and the angle ψ are related to angles φ and θ as
r=SAD sin(φ), (3)
ψ=θ+φ−π/2. (4)

Using these two relations, the ray can be reparametrized as

$$\begin{matrix}SAD sin ( ϕ ) = x cos ( θ + ϕ - \frac{π}{2} ) + y sin ( θ + ϕ - π / 2 ) , & ( 5 ) \\ z = k \frac{θ}{2 π} . & ( 6 )\end{matrix}$$

Using the above parametrization of the ray, the equation 1 can be rewritten as:

$${I}_{d} = {I}_{0} {ⅇ}^{- \int {\int }_{{R}^{2}} μ ( x , y , {z}_{t} ) δ ( SAD sin ( ϕ ) - x sin ( θ + ϕ ) - y sin ( θ + ϕ ) ) ⅆ x ⅆ y} , \qquad ( 7 )$$

Finally, the projection, i.e. the value measured by the detector is

$$\begin{matrix}{d}_{p} ( θ , φ ) = ln \frac{{I}_{0}}{{I}_{d}} \\ = - \int {\int }_{{R}^{2}} μ ( x , y , {z}_{t} ) \\ δ ( SAD sin ( ϕ ) - x sin ( θ + ϕ ) - y sin ( θ + ϕ ) ) ⅆ x ⅆ y .\end{matrix} \qquad ( 8 )$$

The preceding equation defines the sinogram dₚ(θ,φ) as obtained from the tomography scanner, except that sinograms are discrete i.e. sampled on a regular lattice of points. Reconstruction of CT slices consists in recovering μ from the sinogram. The present invention does not use projection data to recover slices. Instead, the information about the seeds is extracted from the sinogram and the seeds are reconstructed using this information only as described below.

Projection of the Seeds

The first step towards reconstruction is to model the image of a single seed in the sinogram. For this purpose, the seeds are modeled as infinitely thin, short line segments. Even though the real seeds have finite radius (typically 0.8 mm), this assumption holds well since the resolution of the detector is about 1 mm. Also, the length of a seed is much larger (typically 5 mm) than its diameter.

Let us assume that the only object between the x-ray tube and the detector is a single straight line L defined as:

$$L ( t ) = [ \begin{matrix}x ( θ ) \\ y ( θ ) \\ z ( θ )\end{matrix} ] = [ \begin{matrix}{x}_{0} + {k}_{x} θ \\ {y}_{0} + {k}_{y} θ \\ k θ\end{matrix} ] \qquad ( 9 )$$

where k denotes the pitch, i.e. the relation between the position of the table and the angle θ as explained earlier. Note that the above parametrization of the line cannot represent lines perpendicular to the Z-axis. This is a special case and will be discussed later.

The sinogram for the line L represents a single curve whose equation is:

$$ϕ = arctan \frac{- sin θ ( {x}_{0} + {k}_{x} θ ) + cos θ ( {y}_{0} + {k}_{y} θ )}{cos θ ( {x}_{0} + {k}_{x} θ ) + sin θ ( {y}_{0} + {k}_{y} θ ) + SAD} \qquad ( 10 )$$

This equation can be easily obtained by first translating the coordinate system for SAD in the direction of X-axis and then applying the rotation for angle θ about the axis Z. In the real sinogram, the image (trace) of a line (seed) projection is defined as a set I={(θᵢ,φ)|i=1, . . . ,N} of N pairs (θᵢ,φ) of coordinates that satisfy the above equation.

To illustrate the principle of the reconstruction, let us assume that the set I has been obtained from the sinogram. The reconstruction problem can be stated as follows: Given the image I={(θᵢ,φ)|i=1, . . . ,N} of a single seed, the reconstruction problem is the problem of recovering the seed orientation and position from I, i.e. parameters of the line defined in Eq. 9.

This problem can be solved as a least-square problem by first rewriting Eq. 10 as (tan φ cos θ+sin θ)(x₀+θkₓ)+(tan φ sin θ−cos θ)(y₀+θk_{y})=−SAD tan φ. (11) Note that in Equation 10, the denominator is always positive and non-null since SAD is always larger than the distance of the seed to the rotation axis. Thus the whole equation can be multiplied by the denominator.

Each sample point (θᵢ,φᵢ) εI has to satisfy the preceding equation, which gives an overdetermined system of N equations with 4 unknowns. The system of equations can be written in matrix notation as:

$$[ \begin{matrix}{a}_{1} & {b}_{1} & {c}_{1} & {d}_{1} \\ {a}_{2} & {b}_{2} & {c}_{2} & {d}_{2} \\ ⋮ & ⋮ & ⋮ & ⋮ \\ {a}_{N} & {b}_{N} & {c}_{N} & {d}_{N}\end{matrix} ] [ \begin{matrix}{x}_{0} \\ {k}_{x} \\ {y}_{0} \\ {k}_{y}\end{matrix} ] = [ \begin{matrix}- SAD tan {ϕ}_{1} \\ - SAD tan {ϕ}_{2} \\ ⋮ \\ - SAD tan {ϕ}_{N}\end{matrix} ] \qquad ( 12 )$$

where
aᵢ=tan φᵢ cos θᵢ+sin θᵢ
bᵢ=θᵢ(tan φᵢ cos θᵢ+sin θᵢ)
cᵢ=tan φᵢ sin θᵢ−cos θᵢ
dᵢ=θᵢ(tan φᵢ sin θᵢ−cos θᵢ)
i.e. A x=B (13)

The least-squares solution of this system is found by computing the pseudoinverse A+ of the matrix A as:
x=A⁺B (14)

Now the main advantage of the reconstruction from sinograms becomes apparent. When reconstructing from CT slices, the number of samples for each seed is low (typically 2 slices, 10 pixels on each slice) considering the number of parameters to be estimated, the level of noise and artifacts. In a sinogram, there is typically 1000 samples for each seed, which means that the procedure described above should yield very accurate results since there are only four parameters to be found.

Another advantage of the sinograms over CT images is that they allow detecting closely spaced seeds. An example of such a configuration of seeds is shown in FIG. 5 where the distance between the seeds is 0.8 mm. Due to the artifacts, these seeds might appear connected in the CT slice. On the other hand, the raw tomography data is artifacts free and distinguishing seeds that are very close together becomes much easier. As shown in the sinogram in FIG. 5 (real tomography data) individual traces for these three seeds can be easily separated.

Preliminary Implementation

The first step of the reconstruction is the detection of traces of seeds in the sinogram. Seed traces provide the samples required for the least squares fitting. Once the samples are obtained, the position and the orientation of the seeds are estimated. To improve robustness of the algorithm, the detection and the estimation of seed positions and orientations are iterated. A preliminary reconstruction procedure is presented.

Seed Detection

As explained above, the projection of a single seed is a bright sinusoidal curve, as shown in FIG. 5. Therefore, the most appropriate approach for the detection is to apply an algorithm for the detection of curves only. The algorithm proposed by C. Steger in “An unbiased detector of curvilinear structures”, published in 1998 in IEEE Transactions on Pattern Analysis and Machine Intelligence was used for the detection of curvilinear structures in grey-scale images. The algorithm estimates the positions of curve points with sub-pixel accuracy, which is typically 10% of the pixel size.

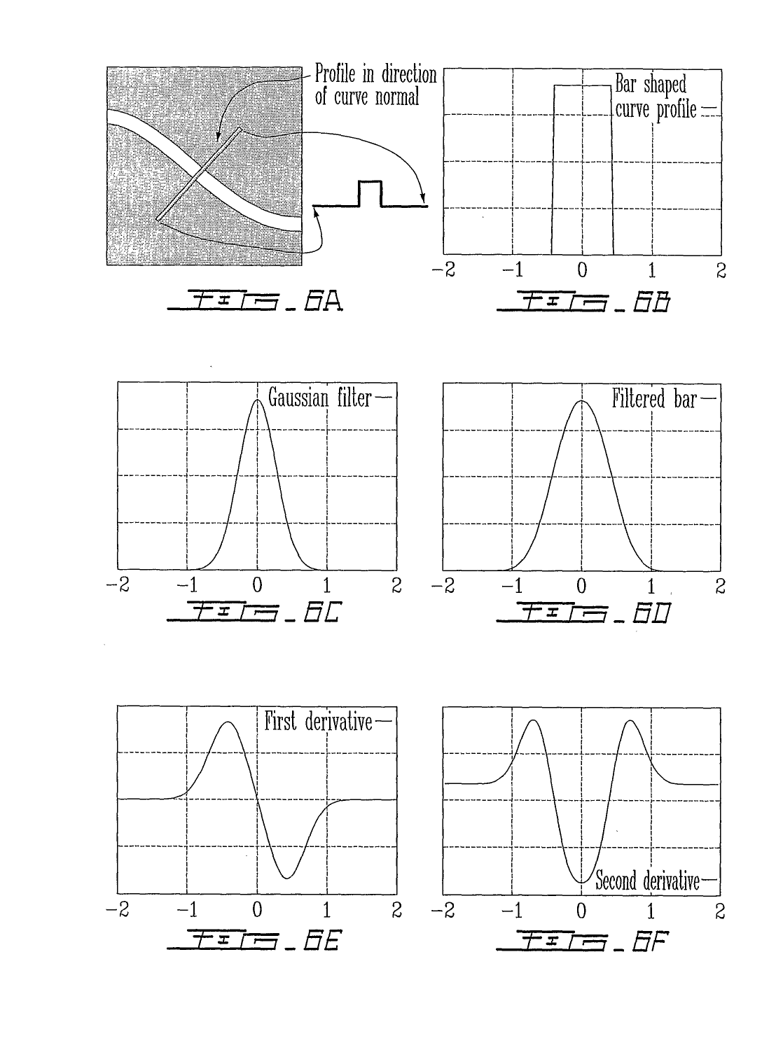

In this algorithm, a curve in the image is modeled as a structure that exhibits, ideally, a bar-shaped profile in the image (in the direction of the normal on the curve), as illustrated in FIG. 6a. In principle, the detection consists in detecting the points where the image profile is bar-shaped, while the estimation of the position consists in estimating the center of the bar.

Ideally, a curve (FIG. 6a) in an image has a bar-shaped profile in the direction of its normal. Filtering the bar profile (FIG. 6b) by convolving it with a Gaussian filter (FIG. 6c) yields a smooth function (FIG. 6d) with a well defined maximum. The maximum corresponds to the center of the bar and can be identified as the point where the first derivative (FIG. 6e) vanishes and where the second derivative (FIG. 6f) takes a large negative value.

One way to estimate the center of a curve in the direction of the normal is to look for the maximal grey scale value. However, bar shaped curves do not have a maximum value at their center. But, convolving the bar-shaped profile with a smooth function, such as a Gaussian filter, results in a function that is smooth and has a well defined maximum that represents the center of the bar. This allows a simple definition of the detection criteria: The points on a curve are points where the first directional derivative (in the direction of the normal) vanishes while the second directional derivative has a large negative value. The position of the center can be obtained by approximating the profile with a second order Taylor polynomial and computing the point where the first derivative is zero.

The only remaining problem is how to estimate the direction of the normal at each pixel. If a point (pixel) is on the curve then the largest change in the image is in the direction perpendicular to the curve. Since the curve shape changes slowly, in the direction of the tangents, grey-scale values change very little. Therefore the normal can be estimated as the direction in which the derivative takes the maximum.

In summary, the algorithm first convolves the image z(x,y) with the following filters:
g_{x,σ}(x,y)=g_{σ}(y)g′_{σ}(x) (16)
g_{y,σ}(x,y)=g′_{σ}(y)g_{σ}(x) (17)
g_{xx,σ}(x,y)=g_{σ}(y)g″_{σ}(x) (18)
g_{xy,σ}(x,y)=g′_{σ}(y)g′_{σ}(x) (19)
g_{yy,σ}(x,y)=g″_{σ}(y)g_{σ}(x) (20)

where

$$\begin{matrix}{g}_{σ} ( t ) = \frac{1}{\sqrt{2 π} σ} {ⅇ}^{- \frac{{t}^{2}}{2 {σ}^{2}}} , & ( 21 ) \\ {g}_{σ}^{′} ( t ) = \frac{- x}{\sqrt{2 π} {σ}^{3}} {ⅇ}^{- \frac{{t}^{2}}{2 {σ}^{2}}} , & ( 22 ) \\ {g}_{σ}^{″} ( t ) = \frac{{x}^{2} - {σ}^{2}}{\sqrt{2 π} {σ}^{5}} {ⅇ}^{- \frac{{t}^{2}}{2 {σ}^{2}}} . & ( 23 )\end{matrix}$$

The results are partial directional derivatives
rₓ(x,y)=z(x,y)*g_{x,σ}(x,y), (24)
r_{y}(x,y)=z(x,y)*g_{y,σ}(x,y), (25)
rₓₓ(x,y)=z(x,y)*g_{xx,σ}(x,y), (26)
r_{xy}(x,y)=z(x,y)*g_{xy,σ}(x,y), (27)
r_{yy}(x,y)=z(x,y)*g_{yy,σ}(x,y). (28)

that are used to estimate the normal at each pixel from the Hessian matrix H

$$H ( x , y ) = ( \begin{matrix}{r}_{xx} & {r}_{xy} \\ {r}_{xy} & {r}_{yy}\end{matrix} ) \qquad ( 29 )$$

The normal is the eigenvector n=[nₓ,n_{y}]^{T} associated with the largest eigenvalue. The position of the curve center along the normal (point where the first derivative in the direction of the normal vanishes) p=[pₓ,p_{y}]^{T} (relative to a pixel) is given as p=t n, where

$$t = \frac{{r}_{x} {n}_{x} + {r}_{y} {n}_{y}}{{r}_{xx} {n}_{x}^{2} + 2 {r}_{xy} {n}_{x} {n}_{y} + {r}_{yy} {n}_{y}^{2}} \qquad ( 30 )$$

A pixel is said to be on the curve if pₓ ε [−0.5,0.5] and p_{y} ε [−0.5,0.5] and if the maximum eigenvalue of the Hessian matrix is above a specified threshold. The parameter σ used in the filters is determined relative to the expected line width w as

$$σ \leq \frac{w}{\sqrt{3}} \qquad ( 31 )$$

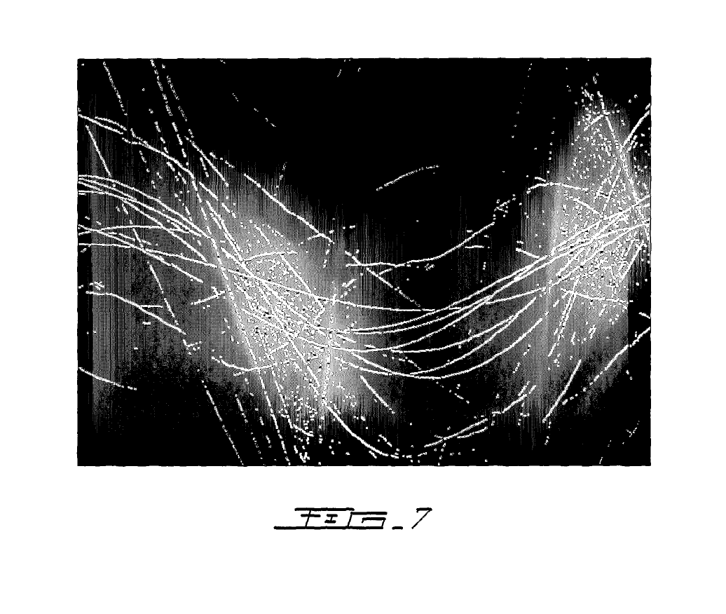

An example of the detection is shown in FIG. 7. FIG. 7 shows an example of detected curvilinear structures in a sinogram. As expected, the algorithm did detect the sinusoidal traces of the seeds. However, there are two new problems to be solved. First, the algorithm detects a considerable amount of noise in lateral views. For those views, the scanner amplifies the signal, and thus the noise, to compensate for the large attenuation of pelvic bones. Also, thin bone structures that leave curvilinear traces in the sinogram are detected as well. Secondly, even though the algorithm detects most of the pixels belonging to seeds, those pixels are not grouped so that each group contains pixels belonging to a single seed. This means that it is not known whether a detected pixel belongs to seeds or noise and, if the points belong to a seed, it is not known to which seed it belongs.

Clearly, the solution for these two problems has to be very robust in order to tolerate the large amount of noise as shown in FIG. 7. Similar problems in the field of image processing are usually solved using the Hough transform which is considered a very robust method. Hough transforms were originally developed for the detection of lines in images, but can be generalized for the detection of arbitrary shapes whose geometry is known. Since the traces of seeds in a sinogram have a known shape (see Eq. 10) the Hough transform can be applied to detect them. To separate seed traces from noise and to group detected curves, a method named Sliding Slice was developed.

Sliding Slice

To illustrate the principle of seed detection, assumption that the sinogram is obtained from a single rotation of the gantry at a fixed position of the table, i.e. zₜ=const is made. This means that the x-ray source and the detector array are all in a single plane, denoted as Π. Furthermore, it is assumed that the seeds are not perpendicular to the scanning axis Z. As mentioned earlier this is a special case that is considered separately.

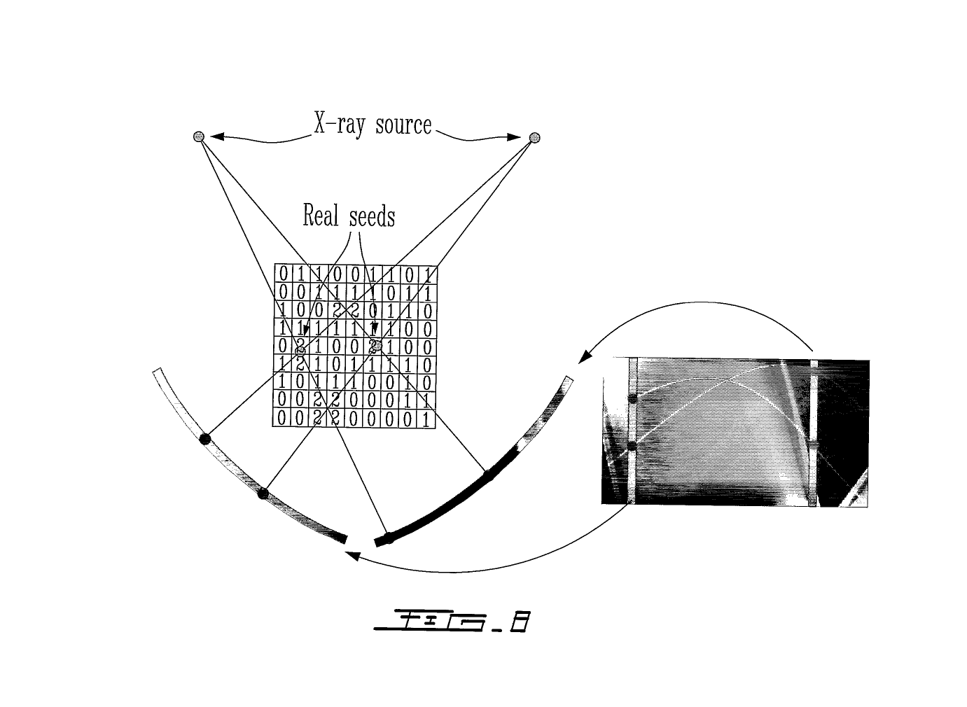

For each position of the gantry, the x-ray source and the detector can be connected where a point has been detected in the previous step, as shown in FIG. 8.

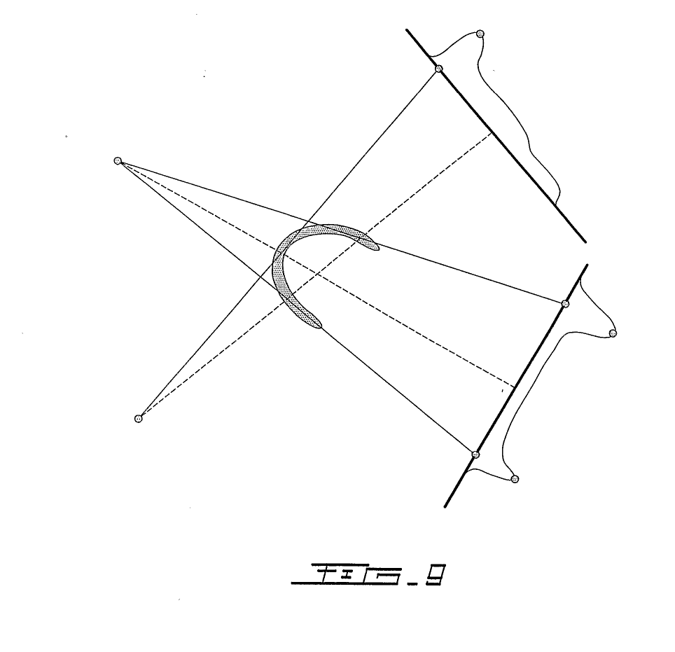

Let the function A:R²→N, referred to as an accumulator, define the number of lines passing through each point in the plane Π. As illustrated in FIG. 8, all lines belonging to a single seed pass through a single point that represents the center of the seed. At these points, the value of the accumulator is high, since a large number of lines pass through them. On the other hand, noise and detected traces of bones and other structures do not create high values in the accumulator, since they do not belong to a point-like structure and thus their lines do not intersect in a single point, as illustrated in FIG. 9.

FIG. 8 shows the principle of the Hough transform for the seed detection method. Detected points are back-projected along the lines that connect them with the x-ray source. Each point in the accumulator contains the number of lines passing through it. All lines belonging to a single seed intersect in a single point. Thus the seeds are detected as the points with high accumulator value. In this example, the accumulator is discrete and each point (pixel) is square of non-zero size. Thus, in some cases, more than one pixel around the real seeds have large value. Also in this example there are ambiguities, i.e. four seeds can be detected instead of two. By adding more views, these ambiguities disappear.

FIG. 9 shows the curve detection algorithm tuned to detect only curvilinear structures (whose profile is bar shaped) in a sinogram. Thus, only very thin structures can be detected. Since the bones and tissues have lower attenuation than metallic seeds, the length of the ray segment that passes through such structures has to be relatively large in order to create a peak in the sinogram and be detected. If that happens, back-projected lines will not intersect in a single point, and thus will not be detected by the Hough transform.

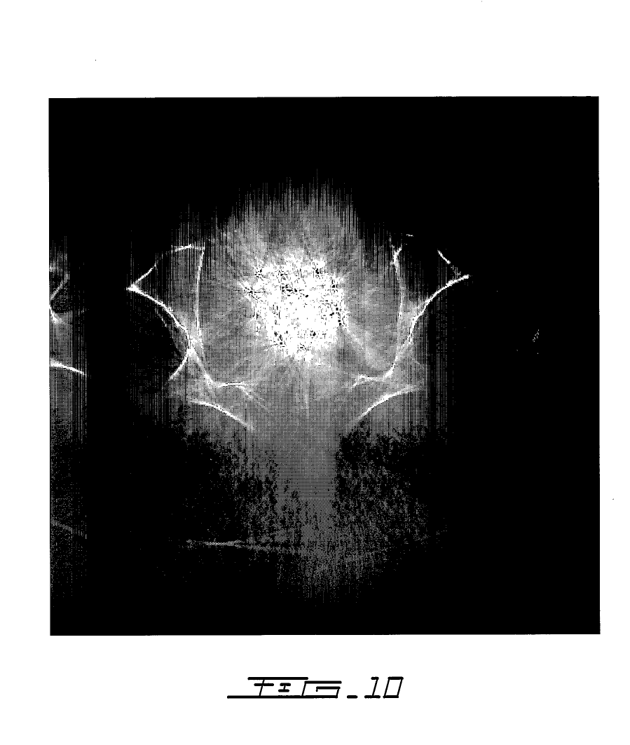

Therefore, by thresholding the accumulator, the seeds can be detected and separated from the noise. An example of the accumulator is shown in FIG. 10. This accumulator represents the Hough transform of the sinogram for a single 360 degrees rotation. Each (point) pixel encodes the number of lines passing through it. Each line connects the x-ray tube and the detector where a curve has been detected.

The sinogram is obtained from viewpoints that have different positions along the scanning axis. If the seeds are not parallel to this axis, their back-projected lines do not intersect in a single point. However, since the accumulator is discrete, its resolution can be chosen to be relatively large with respect to the length of seeds (approximately 0.25 of the seed length). By doing so, the accumulator will have high values in small regions around the true seed center rather than in a single point. Therefore, thresholding the accumulator results in blobs (connected groups of pixels) as shown in FIG. 10. It should be noted that blobs are inevitable and appear even if the seeds are parallel to the scanning axis, as shown in FIG. 8. This is a consequence of using a finite resolution accumulator (In practice, the accumulator is a discrete, rectangular lattice of points, and represented as a 2D bitmap image (as in FIG. 10). The accumulator is computed by back-projecting detected seed traces using Bresenham's algorithm) as well as scanner miscalibration and other mechanical imprecision.

Once the seeds are detected for one position of the accumulator, it is advanced by one in the direction of θ in the sinogram and the whole procedure is repeated (hence the name sliding slice). This way one slice (accumulator) is obtained for each position of the gantry along the scanning axis yielding typically 50,000 accumulator slices. Obviously, complete recomputation of the accumulator for each position of the gantry is long. This problem is solved by incrementally computing the accumulator as follows.

To describe the sliding slice transform more formally, let the set P={pᵢ=(θᵢ,φᵢ)| i=1, . . . ,N} denote the set of all detected points in the sinogram, and let Θ={θᵢ|i=1, . . . M}, θᵢ<θᵢ₊₁ ∀i ε [1, . . . M−1] denote all M positions of the table. Backprojection Aₚ_{ᵢ} of a single point pᵢ=(θᵢ,φᵢ) is defined as:

$${A}_{{p}_{i}} ( x , y ) = \{ \begin{matrix}1 & if ( x , y ) ∈ {L}_{{p}_{i}} \\ 0 & otherwise\end{matrix}$$

where L_{θ}_{ᵢ}_{,φ}_{ᵢ} denotes the line connecting the point (θᵢ,φᵢ) and the x-ray source as defined in Eq. 5. By assuming that there is K table positions in each full rotation, the first accumulator A₁ is computed by back-projecting set of N₁ points

$${P}_{1} = \{ ( {θ}_{i} , {φ}_{i} ) \vert {θ}_{i} ∈ [ {θ}_{1} , … , {θ}_{K} ] \} , i . e . {A}_{1} ( x , y ) = {\sum }_{{p}_{i} ∈ {P}_{1}} {A}_{{p}_{i}} ( x , y ) ,$$

Similarly, the second accumulator A₂ is computed by back-projecting set of N₂ points P₂₁=(θᵢ,φᵢ)|θᵢ ε [θ2, . . . ,θ_{K+1}]}, and so on.

To incrementally compute the accumulator, note that the accumulator is obtained by summing back-projections for individual points and that two subsequent accumulators Aᵢ and Aᵢ₊₁ differ very little: Aᵢ₊₁ does not contain points Q={(θᵢ,φᵢ)|θᵢ=θ₁} whose theta coordinate is θᵢ while Aᵢ does not contain points R={(θᵢ,φᵢ)|θᵢ=θ_{i+K}} whose theta coordinate is θ_{i+K}. In other words the two accumulators are obtained from approximately 99.9% of the same points. Therefore, the computation time can be reduced by several orders of magnitude if Aᵢ₊₁ is computed from Aᵢ instead of recomputing it from scratch. The accumulator Aᵢ₊₁ is obtained from Aᵢ as

$${A}_{i + 1} ( x , y ) = {A}_{i} - {\sum }_{{p}_{i} ∈ Q} {A}_{{p}_{i}} ( x , y ) + {\sum }_{{p}_{i} ∈ R} {A}_{{p}_{i}} ( x , y )$$

Since the above transformation yields blobs that resemble blobs obtained by thresholding CT images, one might argue that it is not an improvement over the use of CT images. However, this is exactly where the power of this approach is hidden: for each detected pixel in the accumulator, it is possible to find out what points in the sinogram generated it. If two pixels belong to the same seed, then they will be generated by the same detected points in the sinogram. Therefore, those pixels can be easily grouped together. This makes the task of breaking the blobs into separate seeds much easier when compared to thresholded CT images where no additional information is available. It should also be noted that the transform described above is used for detection only and not for the estimation of the seed position and orientation as in methods using CT images. Furthermore, the CT images are obtained at discrete positions with a distance of 1 to 5 mm; if the accumulator is considered as an equivalent of a single CT image then this present method reconstructs three orders of magnitude more images, thus more information for each seed.

Space-Time Analysis

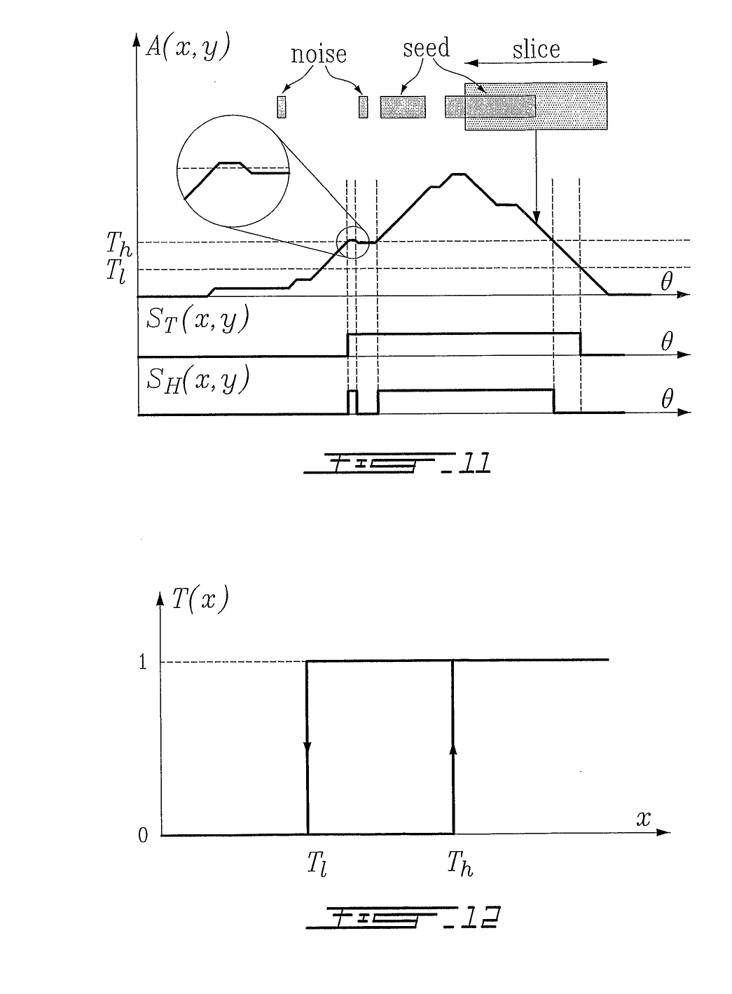

From the above discussion it is clear that, while positions of the seeds have been detected in the image plane, their position along the scanning axis is still unknown. To solve this, the state of each pixel in the accumulator is tracked as the accumulator advances. As illustrated in FIG. 11, the value of a single accumulator point (x,y) is small if the sliding slice does not intersect the seed. If it does, the value increases until the slice passes the seed.

If the value of each accumulator pixel is tracked in time (its position along the Z axis as the accumulator advances) then by imposing a threshold, the seed position can be found along the Z axis. The position of the seeds is the center of a continuous interval where A(x,y) is larger than the threshold. However, due to the noise, the accumulator value can oscillate around the threshold giving rise to non-continuous regions. This is easily solved by using a hysteresis threshold as illustrated in FIG. 11. FIG. 11 shows the detection of the position of the seed along the scanning axis. The upper diagram shows the value A(x,y) of a pixel (x,y) in the accumulator. As the slice slides (shaded region) over a seed the value of the accumulator increases. By setting a threshold on this value, the position of the seed can be estimated. Sₜ(x,y) indicates the presence of a seed and is obtained by thresholding A. Due to the noise this value can oscillate, thus not giving continuous intervals for which the accumulator value is higher than the threshold. The problem can be solved by using two thresholds, i.e. a hysteresis threshold. As the bottom diagram S_{H}(x,y) shows, the obtained region is not sensitive to the oscillations due to the noise.

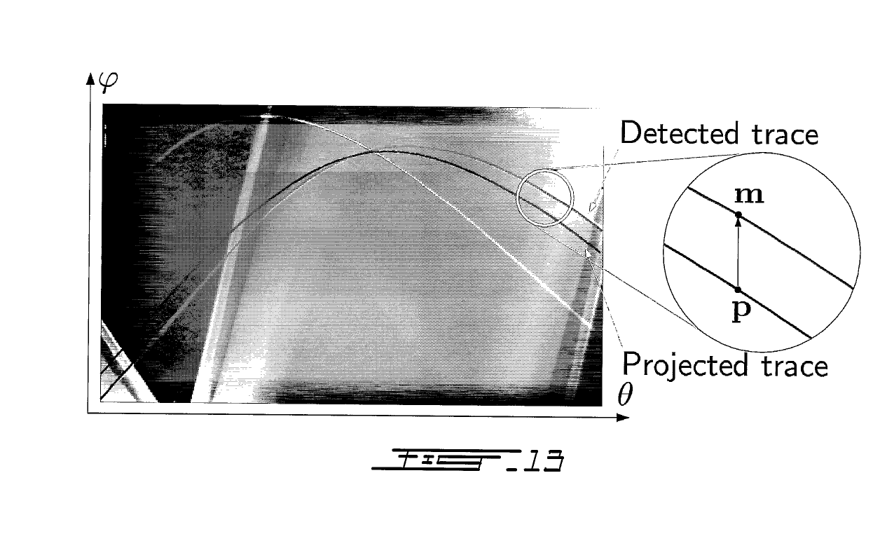

The transfer function of the hysteresis threshold is shown in FIG. 12. The transfer function T gets a high value when the signal passes the high threshold Tₕ. The function falls back-to zero when the signal falls below Tₗ.

Note that two or more seeds can be aligned so that they are detected continuously as a single seed, but only if the seeds are parallel (or almost) to the scanning axis in which case they are easily separated by verifying the length of the intervals. Since the length of seeds is known, intervals longer than a single seed can be easily split in two or more parts.

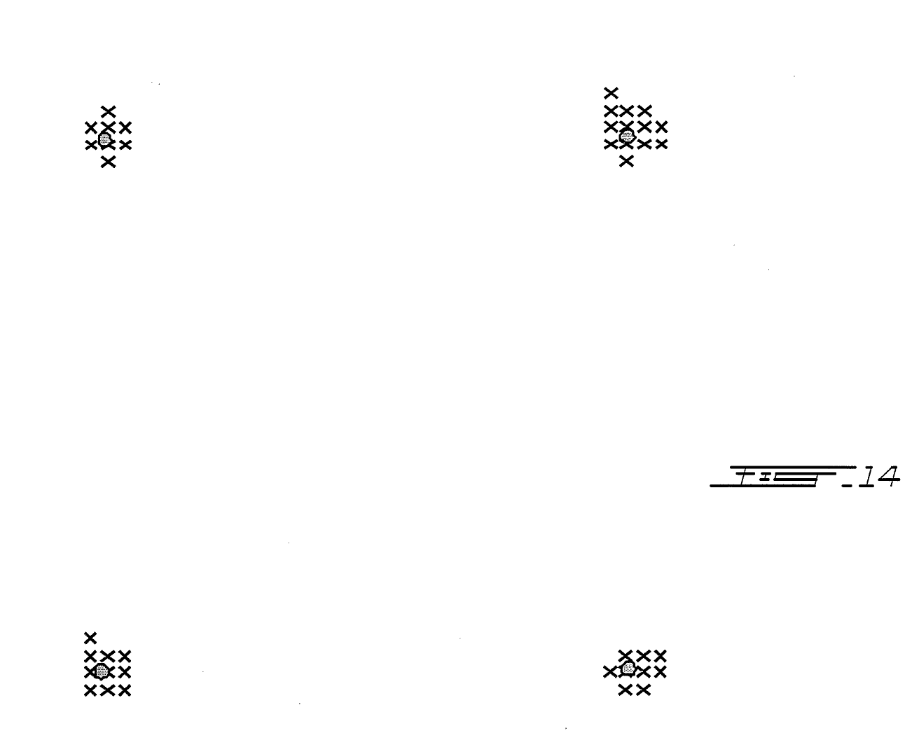

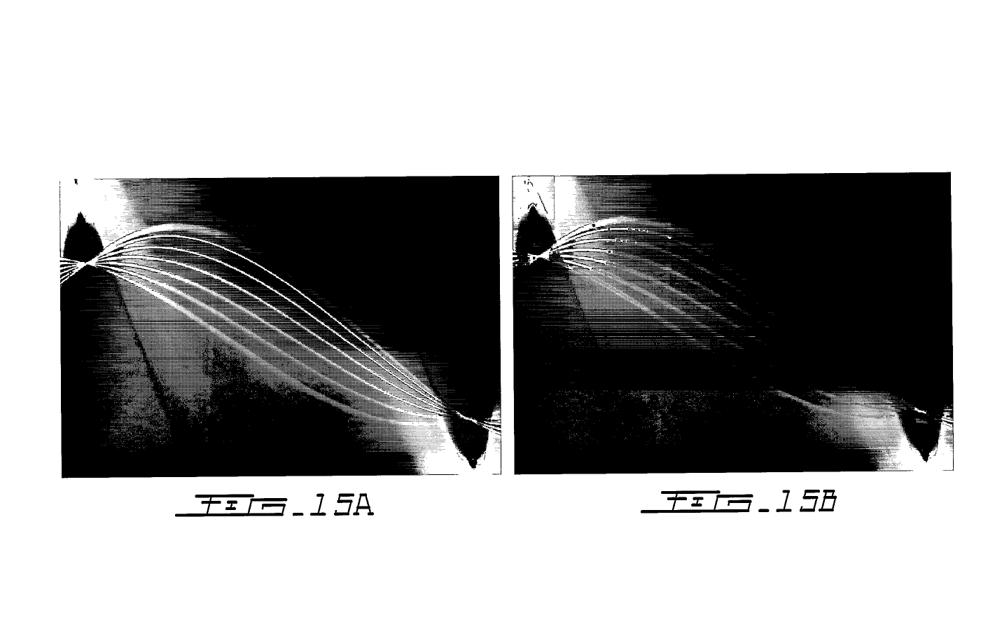

Finally, one obtains a set of points, detected in the accumulator with all three coordinates. As mentioned earlier, detected points are in blobs thus two or more points might represent the same seed. The final step of the reconstruction procedure is to detect these redundant points and reduce blobs to a single point. To do so, all points are projected back into the sinogram. The length of the projection in the sinogram is 50% larger than the real seed length lₛ. The projection is centered at the seed center along the Z-axis. In other words, if the position of the seed center is zₛ then the projection is computed for the interval [zₛ−0.75 lₛ, zₛ+0.75 lₛ]. Then, the projected traces are matched to the closest detected points in the sinogram while initially assuming that the seed is parallel to the Z-axis. From the matched points, the position and the orientation of the seed are estimated. Then, the whole procedure is repeated until the algorithm converges. The centroid of a matched point is taken as the position of the seed along the Z-axis. Finally, the points whose position differs less than the size of pixel in the accumulator are grouped together and they represent a single seed. This procedure is illustrated in FIG. 13. FIG. 14 shows four blobs and the position of each point after the above procedure.

As shown in FIG. 13, to reduce blobs detected in the accumulator to a single point, each point in the blob is projected back into the sinogram. The resulting trace does not overlap perfectly with the detected trace for two reasons. First, initially seeds are assumed parallel to the Z-axis which is not the case in general. Second, none of the points (or at most one) in the blob is in the exact center of the seed. Then, each point p of the projected trace is matched with the closest point m in the detected trace in the direction of axis φ. The set of matched points is used to estimate the new position and the orientation of the seed. Gradually, the estimate is improved and the procedure converges.

As shown in FIG. 14, thresholding the accumulator yields several points for each seed instead of a single point. By projecting each point in the blob back to the sinogram and by matching the resulting trace with the detecting one, the points that belong to a single seed all converge to a single point. Initial points in blobs are indicated as crosses while their final position is shown as a circle. Since all points in a single blob were generated by the same detected trace in the sinogram they all converge to the same point. Thus each blob is reduced to a single point.

Summary of the Overall Method

The overall reconstruction procedure is summarized below with reference to FIG. 17, a flow chart of the main steps. First, acquisition of the sinogram, that is, raw tomography data, is carried out by the scanning device (see equation 8). Detection and estimation of the curves in the sinogram is done by the seed detector (see the paragraph immediately preceding equation 31) to obtain detected curves. The Hough transform is then computed using the detected curve points (see FIG. 8 and its description) and outputs an accumulator. The next main step is thresholding the Hough Transform accumulator in order to obtain blobs representing seeds (see the paragraph beginning with “The sinogram is obtained from viewpoints” at page 18 of the present description). These blobs are candidate seed points detected in the accumulator. Back projection of each pixel in each blob to the sinogram is then carried out to obtain projected points, a matching of the projected curve against the detected curves is done and generates matched points, the parameters representing seed position and orientation are computed from the matched points and the steps of projecting, matching and computing are iterated until convergence (see the paragraph beginning with “Finally, one obtains a set of points” at page 21 of the present description). Finally, coincident seeds (having similar positions) are grouped together and declared to be a single seed (see the paragraph beginning with “The least-squares solution of this system” at page 12 of the present description). The final set of seed orientations and positions is then obtained.

Results

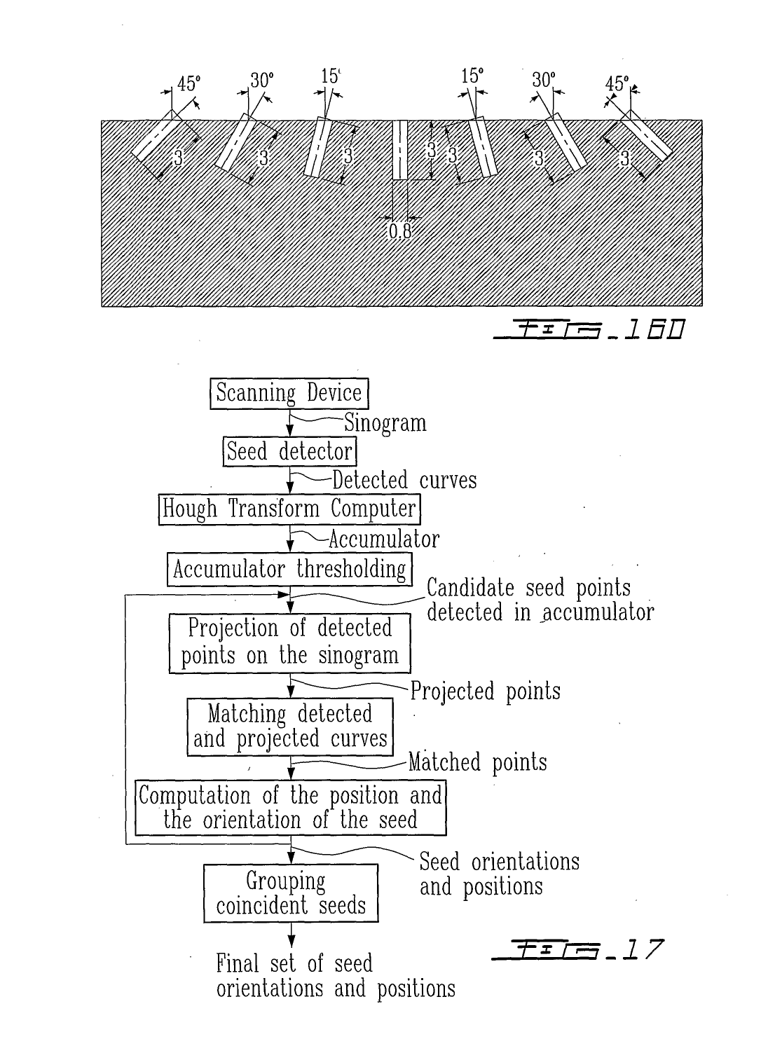

Two phantoms (FIGS. 16a-16d) were used to evaluate the accuracy of the present invention. The first phantom consist of 16 seeds arranged in a regular rectangular 4×4 grid as illustrated in top-view in FIG. 16a. Being intended for the evaluation of positional accuracy, all seeds are parallel as shown in side-view in FIG. 16b. Centers of seeds are coplanar. The second phantom was used to evaluate the influence of the seed orientation on the positional accuracy as well as to evaluate the accuracy of the orientation estimate. For this purpose 7 seeds were arranged in a single row with equal inter seed distance as shown in FIG. 16c. Each seed is rotated by 15 degrees relative to its neighbouring seed as illustrated by the side-view of the phantom in FIG. 16d. The two phantoms have been machined with a tolerance of 0.05 mm for position and 1 degree for orientation.

The position accuracy was determined by measuring the distance between all pairs of seeds and comparing it with the exact distances. The distance was used since it is invariant to the absolute position and orientation of the phantom. The average error was 0.15 mm while the maximal error was 0.45 mm and the standard deviation was 0.11. It should also be noted that the reconstructed phantom appeared scaled down. The most probable cause for this is scanner miscalibration. Due to the lack of documentation for the sinogram files, it was impossible to recover the exact SAD distance as well as the exact orientations of the gantry for each viewpoint. Values used for the reconstruction were the nominal values for the scanner. With proper scanner calibration carried out by one skilled in the art, this error should be reduced by at least 50%. Similarly, the orientation accuracy was measured by computing the angle between all pairs of seeds and comparing it with the exact angles. The average error was 2.57 degrees while the maximal error was 6.29 degrees.

The algorithm as described above was implemented in C++. Without any software optimizations, the total processing time is under 2 minutes on a PC with an AMD Athlon XP 2600 processor. Proper software optimization should reduce the processing time for an order of magnitude, as will be readily understood by one skilled in the art.

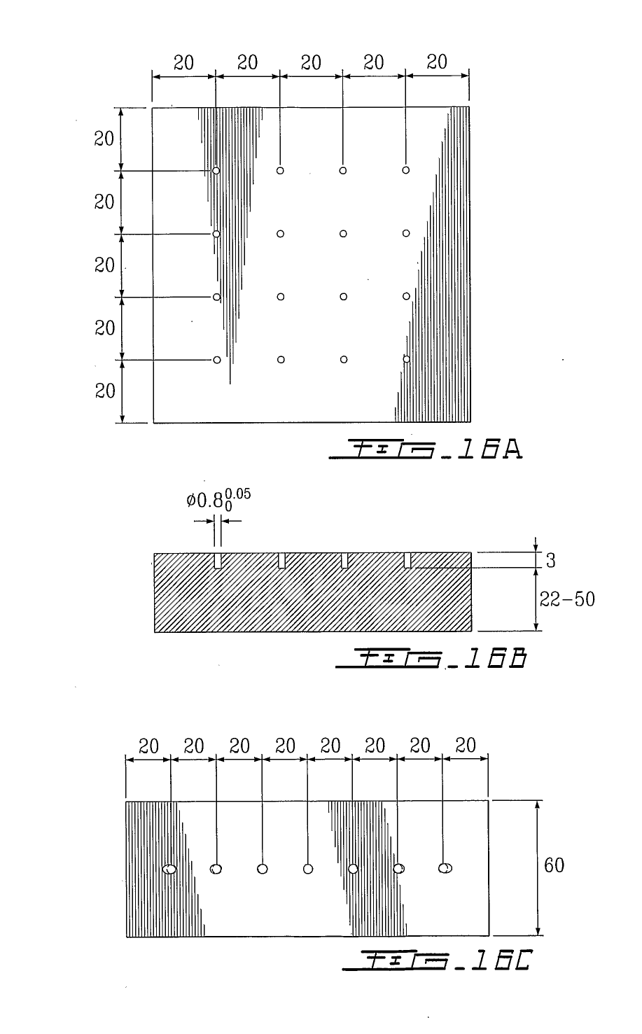

One of the problems that has not been discussed in the present implementation is the variable width of seeds that are not parallel to the scanning axis. As shown in FIG. 15, as the angle between the seed and the scanning axis increases, so does the width of the trace in the sinogram. Since the detection algorithm is tuned to the detection of curves of predetermined width it fails to detect thick traces. This can be solved by detecting the curves twice, using a different σ in Eq. 21-23. However, such configurations are rare in clinical implants with 0.7% of the seeds having an orientation above 75 degrees.

FIG. 15 shows traces of oriented seeds. Due to the finite thickness of the beam, the width of seed traces increases with the angle between the seed and the scanning axis Z. FIG. 15a shows the contrast enhanced sinogram for seven seeds with different orientations. Traces, from top to bottom, belong to seeds whose angles with the Z-axis are 90, 0, 15, 30, 45, 60 and 75 degrees. FIG. 15b shows the result of the detection. Since the algorithm is tuned to specific width of curves, some points are missing for the seed whose orientation is 75 degrees while the seed at 90 degrees is not detected at all. This problem can be solved by detecting the curves twice, using different σ in Eq. 21-23.

The second problem left undiscussed is the estimation of the orientation for those seeds that are perpendicular to the axis-Z. For those seeds, the least-squares fitting does not work since the model in Eq. 9 does not allow those orientations. The problem can be solved by tracking the width of the traces. The orientation of the gantry for which the trace is thinnest coincides with the orientation of the seed. The width of curves can be locally estimated using the same detection algorithm.

For real patients, thin bone structures might be detected along the real seeds. This can be solved by making the detection more robust. Improved, i.e. more robust, Hough transforms exist, where the transform is performed in two passes, the first one as explained before and, in the second one, the accumulator is increased only where a seed has been detected in the previous step, as will be readily understood by one skilled in the art.

The 3D reconstruction method of the present invention can be generalized to allow reconstruction of arbitrary curvilinear structures that leave detectable traces in sinograms, as will be readily understood by one skilled in the art. One potential application is the reconstruction of metallic catheters. It should therefore be understood that the present method can be used in industrial applications where the internal structure of a non-living body or object is sought. Point-like or curvi-linear objects within the body could then be reconstructed using the described method as long as the material of the object within the structure provides sufficient contrast with respect to a material of the structure in raw tomography data.

The embodiment(s) of the invention described above is(are) intended to be exemplary only. The scope of the invention is therefore intended to be limited solely by the scope of the appended claims.

1. A method for three-dimensional reconstruction of an object inserted in one of a living and a non-living body, a material of said object providing sufficient contrast with respect to a material of said body in raw tomography data, said object having one of a point-like shape and a curvilinear shape, the method comprising:
obtaining raw tomography data for an area of said body where said object is inserted;
detecting a trace of said object in said raw tomography data, by extracting points from said trace; and
estimating at least one of a position and an orientation of said object using said points and a known shape of a trace of said object in said raw tomography data.

2. A method as claimed in claim 1, wherein said estimating comprises using a least squares fitting.

3. A method as claimed in claim 1, further comprising iterating said at least one of a position and an orientation of said object until convergence.

4. A method as claimed in claim 1, wherein said estimating comprises finding points where a first directional derivative in a direction of a gradient vanishes while a second directional derivative has a large absolute value.

5. A method as claimed in claim 1, wherein said estimating comprises grouping said points in at least one group representing at least one object by accumulating said points in an accumulator.

6. A method as claimed in claim 5, wherein said accumulating uses a Hough transform.

7. A method as claimed in claim 5, wherein said estimating comprises detecting blobs in said accumulator and projecting said blobs onto said raw tomography data in order to refine grouping of said points to reduce at least one of said blobs into a single point.

8. A method as claimed in claim 1, wherein said body is a human body and a material of said body is bodily tissue.

9. A method for three-dimensional reconstruction of objects inserted in one of a living and a non-living body, a material of said object providing sufficient contrast with respect to a material of said body in raw tomography data, each said objects having one of a point-like shape and a curvilinear shape, the method comprising:
acquisition of raw tomography data;
detection and estimation of the curves in the raw tomography data;
computation of the Hough transform using the detected curve points to obtain an accumulator;
threshold of the accumulator in order to obtain blobs representing seeds;
back projection of each pixel in each blob to the raw tomography data to obtain projected points;
matching of the projected curve against the detected curves to generate matched points;
determination parameters representing seed position and orientation from the matched points;
iterating the steps of back projection, matching and determination until convergence;
grouping together coincident seeds to be a single seed;
generating a final set of seed orientations and positions.

10. A system for three-dimensional reconstruction of an object inserted in one of a living and a non-living body, a material of said object providing sufficient contrast with respect to a material of said body in raw tomography data, said object having one of a point-like shape and a curvilinear shape, the system comprising:
a scanner device for obtaining raw tomography data for an area of said body where said object is inserted;
a trace detector device for detecting a trace of said object in said raw tomography data, by extracting points from said trace; and
an object locator device for estimating at least one of a position and an orientation of said object using said points and a known shape of a trace of said object in said raw tomography data.

11. A system as claimed in claim 10, wherein said object locator uses a least squares fitter device.

12. A system as claimed in claim 10, further comprising an iterator device for iterating said at least one of a position and an orientation of said object until convergence.

13. A system as claimed in claim 10, wherein said object locator device comprises a point extractor device for finding points where a first directional derivative in a direction of a gradient vanishes while a second directional derivative has a large absolute value.

14. A system as claimed in claim 10, wherein said object locator device comprises a point collector device for grouping said points in at least one group representing at least one object by accumulating said points in an accumulator.

15. A system as claimed in claim 14, wherein said point collector device uses a Hough transform device.

16. A system as claimed in claim 14, wherein said object locator device comprises a blob detector device for detecting blobs in said accumulator and projecting said blobs onto said raw tomography data in order to refine grouping of said points to reduce at least one of said blobs into a single point.

17. A system as claimed in claim 10, wherein said body is a human body and a material of said body is bodily tissue.

18. A system for three-dimensional reconstruction of objects inserted in one of a living and a non-living body, a material of said object providing sufficient contrast with respect to a material of said body in raw tomography data, each said objects having one of a point-like shape and a curvilinear shape, the system comprising:
a scanning device for acquisition of raw tomography data;
a seed detector device for detection and estimation of the curves in the raw tomography data;
a Hough Transform calculator device for computation of the Hough transform using the detected curve points;
an accumulator thresholder device for threshold of the accumulator in order to obtain blobs representing seeds;
a back projector device for back projection of each pixel in each blob to the raw tomography data to obtain projected points;
a matcher device for matching of the projected curve against the detected curves to generate matched points;
a parameter determiner device for determination parameters representing seed position and orientation from the matched points;
an iterator device for iterating the steps of back projection, matching and determination until convergence;
a seed grouper device for grouping together coincident seeds to be a single seed;
a seed generator device for generating a final set of seed orientations and positions.
